(19) United States
(12) Patent Application Publication
(10) Pub. No.: US 2018/0104014 A1
(43) Pub. Date: Apr. 19, 2018

(54) SURGICAL DRAPE FOR PATIENT REGISTRATION AND A REGISTRATION METHOD UTILIZING SUCH SURGICAL DRAPE

(71) Applicant: Brainlab AG, Munich (DE)

(72) Inventors: Nadja HEINDL, Munich (DE); Uli MEZGER, Kirchheim (DE); Conrad RODAS, Longmont, CO (US)

(21) Appl. No.: 15/556,437

(22) PCT Filed: Mar. 17, 2015
(86) PCT No.: PCT/EP2015/055558

§ 371 Date: Sep. 7, 2017

(51) Int. Cl. A61B 46/20; A61B 90/90

(52) U.S. Cl. CPC A61B 46/20; A61B 2017/00902; A61B 2046/205; A61B 90/90

(57) ABSTRACT

A surgical drape includes an impervious section having absorbing properties, an incision section including an incise foil, and a transparent section that is sized such that it allows a user to visually observe, through the surgical drape for registration purposes, a substantial part of a patient that is covered by the surgical drape. A method of registering a patient's body part with a pre-acquired image dataset of the body part is provided including providing a surgical drape including the impervious, the incision, and the transparent sections, covering the patient's body part with the surgical drape so as to provide a sterile workplace separated from an unsterile area by the surgical drape, using the transparent section of the surgical drape to visually identify the patient's body part covered by the surgical drape, and approaching and registering the patient's body part with a registration instrument such as for example a pointer instrument.

The present invention relates to a surgical drape for separating a sterile region from an unsterile region during surgery and a method of utilizing such surgical drape during patient registration.

In medical procedures incorporating image-guided surgery (IGS), is desirable to know the position of a specific anatomical structure that can be seen in the medical image of the body of a patient who is to be treated, with respect to the actual patient's body. It is likewise desirable to know the relative position of medical equipment such as medical instruments and a specific anatomic structure that is shown in a medical image of the body of the patient. For both reasons, patient registration procedures have to be performed by means of which the actual body is registered to the at least one medical image. This will allow a medical navigation system to display the position of the anatomical structure relative to the surgical instrument to medical personnel on a monitor.

However, such registration procedures must not compromise sterility of the workplace. Conventional registration methods are therefore performed pre-operatively and prior to draping of the patient. Registration cannot be repeated, for example to restore an initial registration intra-operatively, after the patient has been draped, since anatomical landmarks, structures and artificial fiducials have also been covered by the drape and are therefore inaccessible. WO 2005/002456 A1 suggests to provide a surgical drape having covering elements for reference means that allowed to track the reference means intra-operatively. This, however, does not allow for re-registering the patient intra-operatively, as well.

The present invention provides a surgical drape and a method of registering a patient's body part, that allow to register a patient's body part intra-operatively after it has been covered by a surgical drape prior to surgery.

The inventive surgical drape and the inventive method of registering a patient's body part are defined by the appended independent claims. Advantages, advantageous futures, advantageous embodiments and advantageous aspects of the present invention are disclosed in the following and contained in the subject-matter of the dependent claims. Different advantageous futures can be combined in accordance with the invention wherever technically expedient and feasible. Specifically, a feature of one embodiment which has the same or a similar function to another feature of another embodiment can be exchanged with said other feature. A feature of one embodiment which adds an additional function to another embodiment can in particular be added to said other embodiment.

The surgical drape according to the present invention comprises:
an impervious section (2) having absorbing properties;
an incision section (3) comprising an incise foil; and
a transparent section (4); characterized in that
   the transparent section (4) is sized such that it allows to visually observe through the surgical drape (1) a substantial part of a patient for registration purposes that is covered by the surgical drape (1).

The inventive surgical drape comprises an impervious section having absorbing properties which is already known from prior art surgical drapes and which has a layer configured to absorb body fluids, and a further layer that is configured to provide an impervious barrier preventing any substance to penetrate the impervious section of the surgical drape. For example, the impervious layer may be a layer of the impervious section adjacent to an unsterile section underneath the drape covering the patient, whereas the absorbing section may face towards the sterile workplace. Moreover, the inventive surgical drape comprises an incision section having an incision foil which may have adhesive properties on one side to stick the incision section down on the patient's skin right next to the incision area. Additionally to the impervious section and the incision section, the inventive surgical drape further comprises a transparent section trough which medical personnel can see the patient and medical equipment covered by the drape to such an extend so that a registration procedure can be performed on a body part of the patient. For this purpose, the transparent section allows a surgeon to immediately see any anatomical or artificial landmarks the position of which has to be determined for registration.

The transparent section may therefore be sized such that substantially a whole body part to be registered, particularly a structurally stable body part such as a head, a thigh, a lower leg, an upper arm, a forearm or a pelvis covered by the surgical drape (1) can be visually observed.

According to this aspect, the size of the transparent section corresponds to the size of the body part to be registered. For surgery performed on a human head, the transparent section may therefore be sized such that the whole head can be observed through the transparent section, whereas for knee surgery, for example, the transparent section can be sized such that a substantial part of the thigh as well as a substantial part of the lower leg can be covered by and observed through the transparent section of the surgical drape.

The same applies to the shape of the transparent section which can correspond to the shape of the patient's body part to be treated. For example, a surgical drape used for head surgery can have a transparent section with a round or an oval shape, whereas a drape for operations performed on a human leg may have a transparent section with a longitudinal shape.

For example, the size the transparent section can amount to more than 20% of the overall size of the surgical drape or even to more than 30% of the overall size of the surgical drape.

The transparent section may be positioned relative to the incision section of the surgical drape in any expedient manner. For example, the transparent section and the incision section can form two windows in the surgical drape separated by an impervious section. Another example of the surgical drape may however have an incision section and a transparent section which lie adjacent to each other. A further example of the inventive surgical drape can have an incision section surrounded by a transparent section which in turn forms a window within the surgical drape.

It is evident from the description that the transparent section of the inventive surgical drape may have any form and size that appears to be expedient for a specific surgical procedure to be performed.

Further, the surgical drape can be pre-assembled, for example by gluing the impervious section, the incision section and the transparent section together. Moreover, the surgical drape can have the form of a sheet that extends in two dimensions without having any bulge or pouch.

Another example of the inventive surgical drape comprises fixation means that releasably hold together a compacted area of the surgical drape's transparent section, for example a folded, pleated or rolled area. This will allow, medical personnel to increase the size of the transparent section up to a size that is deemed to be appropriate for a specific procedure performed. For example, the transparent section of the surgical drape can be provided with Velcro-straps or adhesive straps that hold a certain portion of the transparent section together, and may be released in case a lager transparent section is needed.

A further aspect of the present invention relates to a method of registering a patient's body part with a pre-acquired image dataset of the body part, comprising the following steps:
providing a surgical drape comprising a transparent section, particularly a surgical drape as described herein;
covering the patient's body part with the surgical drape so as to provide a sterile workplace separated from an unsterile area by the surgical drape;
use the transparent section of the surgical drape to visually identify the patient's body part covered by the surgical drape; and
approach and register the patient's body part with a registration instrument, particularly a pointer instrument.

As already explained further above, the inventive registering method makes use of a transparent section of a surgical drape that allows any medical personal to visually observe a patient's body part and medical equipment through transparent section of the surgical drape for registration purposes.

Again, it is noted that for registration purposes, it is necessary to see large parts of the patient's body together with natural landmarks of artificial fiducials attached to the body part through the drape, so that these landmarks or fiducials can be approached and palpated fast and easily with a registration instrument.

The inventive method can be performed with an unsterile registration instrument held and moved by a user within an unsterile area. In other words, landmarks or fiducials are palpated with an instrument held under the surgical drape, wherein the user can see the instrument through the transparent section of the surgical drape. In case the instrument is tracked via optical tracking cameras, the surgical drape's transparent section has to be translucent for the wavelengths used by the tracking cameras.

In the alternative, the inventive method can also be performed with a sterile registration instrument held and moved within the sterile workplace, i. e. above the surgical drape. In this case it is only necessary to see substantial parts of the patient's body part together with the landmarks or fiducials through the transparent section of the drape, so that the registration procedure can be performed fast and easily.

The inventive method can also comprise a surface matching procedure for which the surgeon sweeps the tip of a registration instrument over the patient's skin so as to determine its form and position. On the other hand, the registration can be performed by palpating predetermined natural landmarks or artificial fiducials for matching them to corresponding landmarks or fiducials, respectively, which can be identified in an image or an image data set of the patient.

With the large transparent section providing the possibility to observe whole body parts of the patient through the surgical drape, it is evident that the registration procedure can be performed prior to surgery as well as during a surgical procedure, for example as a re-registration. In both cases it is possible to attach artificial fiducials onto the patient's skin before the patient is covered with the surgical drape.

With a surgical drape having a size-adjustable transparent section that can be used for a multitude of surgical procedures requiring different sizes of the transparent section, medical personnel can adjust the size of the transparent section by at least partially releasing the fixation means holding together a compacted area of the transparent section.

In the following, the invention is described with reference to the figures which represent preferred embodiments of the invention without limiting the invention to the specific features shown in the figures.

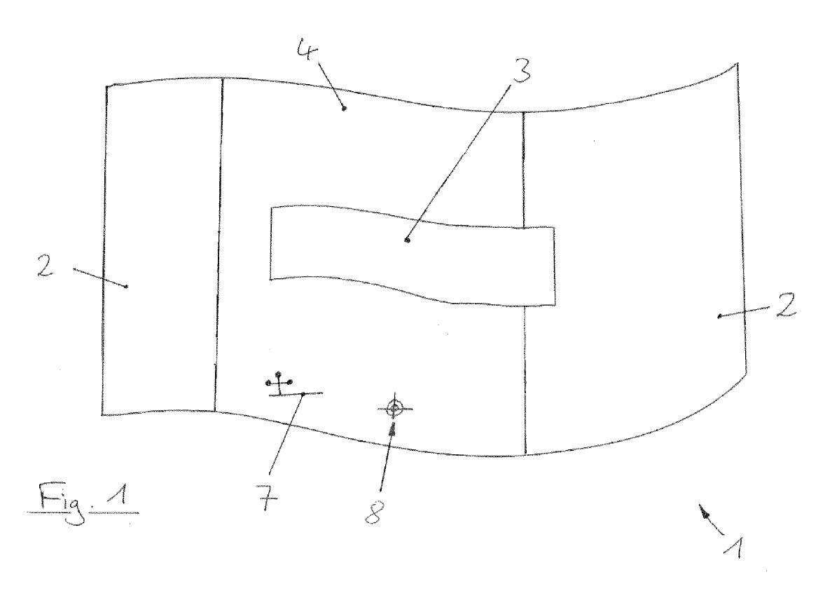

FIG. 1 shows a first embodiment of the inventive surgical drape;

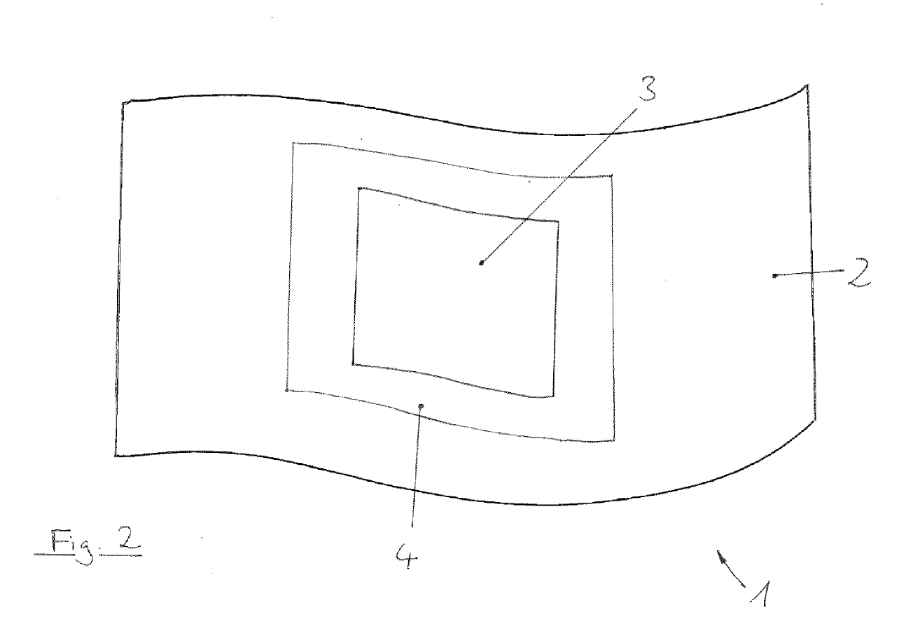

FIG. 2 shows a second embodiment of the inventive surgical drape;

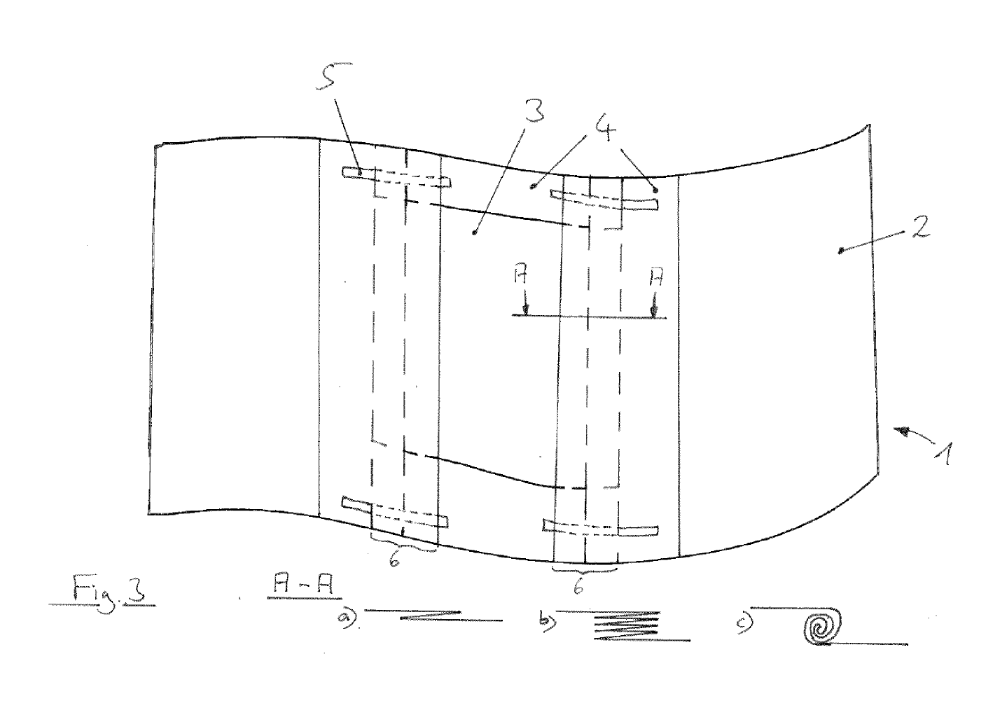

FIG. 3 shows a third embodiment of the inventive surgical drape; and

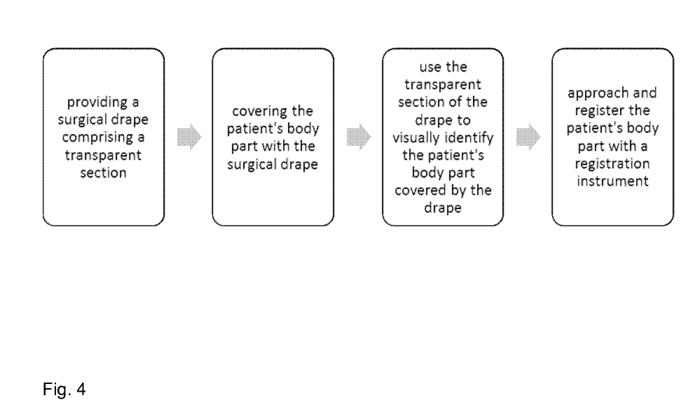

FIG. 4 illustrates a first embodiment of the inventive registration method.

The surgical drape 1 shown in FIG. 1 comprises a transparent section 4 that extends over the whole width of the surgical drape 1 and is flanked on both sides by impervious sections 2. The surgical drape 1 further comprises an incision section 3 that can be adhered to the skin of a patient to be treated. FIG. 1 further shows that the transparent section 4 provides a large area in the vicinity of the incision section 3 that enables medical personnel to visually observe not only the incision area but also large parts of the patient's body. FIG. 1 illustrates that a pointer instrument 7 and an artificial fiducial 8 attached to the patient's skin can be seen through transparent section 4 of the surgical drape 1.

It is important to note that incision section 3 does not necessarily have to be entirely surrounded by the transparent section 4 but may also, at least partially, border directly to the impervious section 2 of the surgical drape 1. Moreover, it is to be noted that the size and shape of the impervious sections 2, the incision section 3 and the transparent section 4 as shown in the Figures are arbitrary and that these sections may have any form or size that appears to be suitable for a specific surgical procedure the surgical drape is designed for.

FIG. 2 shows another embodiment of the inventive surgical drape 1 having an incision section 3 entirely surrounded by a transparent section 4, which in turn is embedded in an impervious section 2.

FIG. 3 shows a further embodiment of the inventive surgical drape 1 with the transparent section 4 extending over the whole width of the surgical drape 1. The transparent section 4 comprises four adhesive straps that hold together two pleated areas 6. Example b) of FIG. 3 shows a cross-sectional view along line A-A through one of the pleated areas 6. The folding edges of the pleated areas 6 as well as the edges of the adhesive straps 5 and the edges of the incision section 3 that are covered by the uppermost layer of the transparent section 4 are shown in broken lines. It is to be noted that the compacted areas 6 can also be formed by a mere double-fold or by coiling up parts of the transparent section, as it is shown by examples a) and c) of the cross-sectional view A-A. The compacted areas 6 of the surgical drape 1 shown in FIG. 3 allows medical personnel to adjust the size of the transparent section 4 according to a specific surgical procedure to be performed.

One embodiment of the inventive registration method is illustrated in FIG. 4. A surgical drape, for example a drape 1 as described herein, having a transparent section 4 is provided and used to cover a patient's body part a surgical procedure is planned to be performed on. After the patient's body part has been draped it can still be seen through the transparent section 4 of the surgical drape 1 along with natural landmarks and artificial fiducials 8 that might be attached to the patient's skin before it was covered by the drape 1. This enables medical personnel to use a registration instrument such as a pointer instrument 7 to palpate the landmarks or fiducials fast and easily, or simply sweeping the tip of the instrument 7 across the patient's skin for surface matching. This can either be done with an unsterile instrument 7 moved underneath the drape 1 or with a sterile instrument moved above the drape 1 within the sterile working place.

1. A surgical drape for covering an associated patient during a procedure on the associated patient, the surgical drape comprising:
an impervious section having absorbing properties;
an incision section comprising an incise foil; and
a transparent section adapted to allow viewing through the surgical drape of a substantial part of the associated patient for registration purposes that is covered by the surgical drape, wherein the incision section and the transparent section lie adjacent to each other and wherein the transparent section is flanked by the impervious section.

2. The surgical drape according to claim 1, wherein the transparent section is sized such that substantially a whole body part of the associated patient covered by the surgical drape can be visually observed.

3. The surgical drape according to claim 1, wherein the size of the transparent section is more than 20% of the overall size of the surgical drape.

4. The surgical drape according to claim 1, wherein the transparent section is sized and/or shaped corresponding to the size and/or shape of the patient's body part to be treated.

5. The surgical drape according to claim 1, wherein the transparent section surrounds the incision section.

6. The surgical drape according to claim 1, wherein the surgical drape has a form of a 2-dimensional plane.

7. The surgical drape according to claim 1, further comprising at least one fixation member releasably holding together a compacted area of the transparent section, the compacted area being a folded, pleated or rolled area.

8. A method of registering a body part of an associated patient with a pre-acquired image dataset of the body part, the method comprising:
providing a surgical drape comprising:
an impervious section having absorbing properties;
an incision section comprising an incise foil; and
a transparent section adapted to allow viewing through the surgical drape for registration purposes of a substantial part of the associated patient that is covered by the surgical drape, wherein the incision section and the transparent section lie adjacent to each other and wherein the transparent section is flanked by the impervious section;
covering the body part of the associated patient with the surgical drape to provide a sterile workplace separated from an unsterile area by the surgical drape;
using the transparent section of the surgical drape to visually identify the body part of the associated patient covered by the surgical drape; and
approaching and registering the body part of the associated patient with a registration instrument.

9. The method of claim 8, wherein the approaching and registering comprises approaching and registering the body part of the associated patient with an unsterilized registration instrument used within the unsterile area for the registering of the body part.

10. The method of claim 8, wherein the approaching and registering comprises approaching and registering the body part of the associated patient with a sterile registration instrument used within the sterile workplace for the registering of the body part of the associated patient.

11. The method of claim 9, wherein the registering comprises palpating and/or sweeping the body part of the associated patient with the registration instrument and/or palpating artificial fiducials attached to the body part of the associated patient.

12. The method of claim 8, further comprising attaching artificial fiducials onto the body part of the associated patient prior to covering the associated patient with the surgical drape.

13. The method of claim 8, wherein the registering of the body part of the associated patient is an initial registration performed prior to surgery.

14. The method of claim 8, wherein the registering of the body part of the associated patient is a follow-up registration performed during a surgical procedure.

15. The method of claim 8, further comprising:
adjusting the size of the transparent section by releasing fixation means of the surgical drape, the fixation means comprising at least one fixation member releasably holding together a compacted area of the transparent section of the surgical drape, the compacted area being a folded, pleated or rolled area.

16. The surgical drape according to claim 2, wherein the transparent section is sized such that a structurally stable body part of the associated patient such as a head, a thigh, a lower leg, an upper arm, a forearm or a pelvis covered by the surgical drape can be visually observed.

17. The method of claim 10, wherein the registering comprises palpating and/or sweeping the body part of the associated patient with the registration instrument and/or palpating artificial fiducials attached to the body part of the associated patient.
